(19) United States
(12) Patent Application Publication
(10) Pub. No.: US 2007/0208210 A1
(43) Pub. Date: Sep. 6, 2007

(54) METHOD AND APPARATUS TO UNLOAD A FAILING HEART

(75) Inventors: Mark Gelfand, New York, NY (US); Howard Levin, Teaneck, NJ (US)

Correspondence Address: NIXON & VANDERHYE, PC, 901 NORTH GLEBE ROAD, 11TH FLOOR, ARLINGTON, VA, 22203, US

(73) Assignee: G&L Consulting, LLC, New York, NY (US)

(21) Appl. No.: 11/680,724

(22) Filed: Mar. 1, 2007

Related U.S. Application Data
(60) Provisional application No. 60/825,033, filed on Sep. 8, 2006.
(60) Provisional application No. 60/743,392, filed on Mar. 2, 2006.

(51) Int. Cl. A61M 1/12

(52) U.S. Cl. 600/16

(57) ABSTRACT

A method and apparatus for treatment of heart failure by reducing LV diastolic volume and pressure by pumping blood out of the LV during diastole. A pump is synchronized to the heart cycle, connected to the apex of the LV and discharging into the right atrium of the heart. A left ventricle to aorta one-way valved conduit with added compliance decreases blood pressure in the aorta and the resistance to the ejection of blood by the heart decreases the energy requirements of the heart.

### CROSS-REFERENCE TO RELATED CASES

This application claims the benefit of U.S. Provisional Patent Application Ser. No. 60/743,392, filed Mar. 2, 2006, and entitled “Valved Conduit with Compliance,” and U.S. Provisional Patent Application Ser. No. 60/825,033, filed Sep. 10, 2006, and entitled “Method and apparatus to Unload the failing Heart.” These provisional applications are both incorporated in their entirety into this application.

### BACKGROUND OF THE INVENTION

The present invention generally relates to implantable devices for treatment of heart failure, and more particularly, relates to implantable blood pumps. The invention may be used to improve the heart muscle performance, reduce pulmonary edema and prevent or reverse heart muscle remodeling. This invention also relates to devices and methods for increasing aortic compliance, and to a second one-way valved conduit from left ventricle to aorta.

Congestive Heart Failure:

Congestive heart failure (CHF) occurs when muscle cells in the heart die or no longer function properly, causing the heart to lose its ability to pump enough blood through the body. Heart failure usually develops gradually, over many years, as the heart becomes less and less efficient. It can be mild, scarcely affecting an individual's life, or severe, making even simple activities difficult. Congestive heart failure (CHF) accounts for over 1 million hospital admissions yearly in the United States (U.S.) and is associated with a 5-year mortality rate of 40%-50%. In the U.S., CHF is currently the most costly cardiovascular disease, with the total estimated direct and indirect costs approaching $56 billion in 1999.

Recent advances in the treatment of CHF with medications, including angiotensin-converting enzyme (ACE) inhibitors, beta-blockers (Carvedilol, Bisoprolol, Metoprolol), Hydralazine with nitrates, and Spironolactone have resulted in significantly improved survival rates. Although many medications have been clinically beneficial, they fall short of clinician's expectations and as a result consideration has turned to procedures and devices as additional and more potent heart failure therapy.

There has been recent enthusiasm for biventricular pacing (pacing both pumping chambers of the heart) in congestive heart failure patients. It is estimated that 30% to 50% of patients with CHF have inter-ventricular conduction defects. These conduction abnormalities lead to a discoordinated contraction of the left and right ventricles of an already failing and inefficient heart. When the right ventricle alone is paced with a pacemaker, the delayed activation of the left ventricle, can also lead to significant dyssynchrony (delay) in left ventricular contraction and relaxation. Because ventricular arrhythmias continue to threaten CHF patients and many antiarrhythmic drugs have unacceptable side effects, a sophisticated implantable cardioverter-defibrillator (ICD) device has shown encouraging results. Biventricular pacing in combination with ICDs demonstrates a trend toward improved survival. Preliminary data in animals and humans using subthreshold (of the type that does not by itself cause heart muscle to contract) stimulation of the heart muscle to modulate cardiac contractility are encouraging and may further enhance the quality of life of CHF patients.

It is also clear that many patients with CHF are not candidates for biventricular pacing or do not respond to this treatment strategy. This also applies to other recent advances and experimental therapies. There is a clear need for new, better therapies that will improve and prolong life of heart failure patients and reduce the burden on the medical system. It is particularly important that these new therapies should not require a major surgery, prolonged stay in the hospital or frequent visits to the doctor's office.

A ventricular assist device (VAD) is a mechanical pump that helps a heart that is too weak to pump blood through the body. It is sometimes referred to as “a bridge to transplant” since it can help a patient survive until a heart transplant can be performed. Some VADs are now used for long-term (destination) therapy in severe heart failure patients who are not candidates for heart transplants. A VAD does not replace the heart. Instead, it works with the patient's own heart to pump sufficient blood throughout the body. The VAD consists of a pump, a control system, and an energy supply. Some VADs rely on a battery for their energy supply; others use compressed air (pneumatic). The energy supply and the control system are located outside the body; the pump can be either inside or outside the body. In a VAD, blood flows from the ventricles into a pump. A left ventricular assist device (LVAD) receives blood from the left ventricle and delivers it to the aorta—the large artery that carries the blood from the heart to the rest of the body. A right ventricular assist device (RVAD) receives blood from the right ventricle and delivers it to the pulmonary artery—the artery that carries blood from the heart to the lungs.

Left Ventricle (LV) enlargement must occur after a large infarct in order to maintain or restore cardiac output in the presence of the loss of the significant amount of contracting muscle tissue. The LV enlargement is necessary to compensate for this loss. In fact, an enlarged ventricle can eject a larger stroke volume, despite unchanged fiber shortening. The disadvantage of dilatation is the extra workload imposed on normal, residual myocardium and the increase in wall tension (according to the LaPlace Law), which represent the stimulus for hypertrophy. If hypertrophy is not adequate to match increased tension, then a vicious cycle will start which determines further and progressive dilatation. The described mechanism explains how an infarct that at the end of the expansion process exceeds certain size is likely to trigger the long-term irreversible sequence of hypertrophy, dilation and chronic heart failure leading to disability and death.

Experimental surgical treatments include approaches to reduce the diameter of the enlarged heart. For example, during the Batista procedure, the surgeon cuts out a piece of the patient's enlarged left ventricular muscle, to reduce the size of the heart. The intention is to restore the size of the left ventricular cavity to normal, improve left ventricular function and reverse congestive heart failure. Other treatments envision surrounding the heart, or a significant portion thereof, with a jacket. An experimental CSD device used to restrain the heart was made by the Acorn Cardiovascular Inc. (St. Paul, Minn.). The Acorn device, a textile girdle or so-called “cardiac wrap,” is wrapped around both the left and right ventricles, thereby preventing further enlargement of the heart.

Benefits exhibited by constraining the heart can be traced down to the relationship between the changing geometry of the heart and the stress in the heart muscle that forms the ventricular wall. The Law of LaPlace says that wall tension is proportional to the product of intraventricular pressure and ventricular radius. Wall tension can be thought of as the tension generated by the heart muscle fibers that results in a given intraventricular pressure at a particular ventricular radius. Therefore, when the ventricle needs to generate greater pressure, for example with the increased afterload (aortic pressure) the wall tension is increased. This relationship also shows us that a dilated ventricle (as occurs after an MI or in dilated cardiomyopathy) has to generate increased wall tension to produce the same intraventricular pressure.

Heart failure is a complex clinical syndrome that can result from any structural or functional cardiac disorder that impairs the ability of the ventricle to fill or eject blood. Each side of the heart is made up of two chambers: the atrium, or upper chamber; and the ventricle, or lower chamber. The atria receive blood into the heart, and the ventricles pump blood out of the heart. Heart failure occurs when either ventricle loses its ability to keep up with the amount of blood flow. Left sided, or left ventricular heart failure, involves the left ventricle. Oxygen rich blood travels from the lungs to the left atrium, and then on to the left ventricle, which pumps the blood to the rest of the body. Because this chamber supplies most of the heart's pumping power, it is thicker than the right ventricle and essential for cardiac output. Blood coming into the left atrium from the lungs may “back up”, causing fluid to leak into the lungs causing pulmonary edema. Ultimately, this may lead to right heart failure. As the right ventricles' ability to pump decreases, the return of blood to the right side of the heart slows down, causing fluid to build up in tissues throughout the body. This excess fluid, or congestion, explains the term congestive heart failure (CHF).

Role of Aortic Compliance:

The aorta is the largest artery in the body. The aorta arises from the left ventricle of the heart, goes up (ascends) a little ways, bends over (arches), then goes down (descends) through the chest and traverses the abdomen to where ends by dividing into two arteries called the common iliac arteries that go to the legs. Anatomically, the aorta is traditionally divided into the ascending aorta, the aortic arch, and the descending aorta. The descending aorta is, in turn, subdivided into the thoracic aorta (that descends within the chest) and the abdominal aorta (that descends within the belly). The aorta gives off branches that go to the head and neck, the arms, the major organs in the chest and abdomen, and the legs. It serves to supply them all with oxygenated blood. The aorta is the central conduit from the heart to the body.

In addition to being a blood conduit, the aorta acts as a compliant tube that buffers and conducts blood ejected from the heart in a pulsatile manner and is the major source of compliance in the entire arterial tree. The classic mathematical model introduced by O. Frank describing pulse wave propagation and arterial mechanical properties assumes that the arterial tree is an elastic chamber (or windkessel) in which, following the ejection by the heart, the diastolic pressure decays exponentially with a time constant that is determined by total arterial resistance and compliance. The simplest definition of compliance is the change in blood volume relative to a given change in distending pressure.

Compliance of the large conduit arteries has been found to be decreased as a result of aging, arterial hypertension, atherosclerosis, diabetes, and heart failure. Changes in the composition of the vessel wall and changes in vessel geometry accompanying these cardiovascular and metabolic disease states are the leading mechanisms explaining a decrease in vascular compliance. A decrease in aortic compliance increases cardiac and vascular load and leads to increases in systolic pressure and pulse pressure, independent risk factors for development of cardiovascular disease.

There is a long standing and well recognized need to increase the compliance of the aorta and reduce the resistance of the aorta and aortic valve in patients with certain forms of heart disease. Proposals were made to reduce blood pressure by absorbing energy of the heart with artificial implanted “compliance” devices.

U.S. Pat. No. 4,938,766 to Jarvik “Prosthetic compliance devices” describes implantable prosthetic devices for increasing arterial compliance and reducing the magnitude of the pressure pulsations in the arterial system. It is hereby incorporated by reference in its entirety.

U.S. Pat. No. 5,409,444 Kensey “Method and apparatus to reduce injury to the vascular system” describes an apparatus and method for reducing peak systolic blood pressure and the rate of change of velocity of the blood flow in a living being by passively absorbing a portion of the blood pressure during systole with an compressible balloon implanted in the aorta.

Although heart failure and hypertension have significantly increased in incidence, there has not been much progress made in implantation of devices to increase or supplement the natural aortic compliance in humans. This should not be explained by the lack of theoretical knowledge, but by the lack of a practical solution that would allow a reasonably simple surgery.

As a result, despite the widespread opinion that increases in aortic compliance may be responsible for facilitation of cardiac ejection and multiple attempts over several decades at implementing this theoretical principle, there has been little progress in translating this belief into a practical therapy.

Apicoaortic Conduits:

Implantation of an additional conduit between the left ventricle and the aorta (an apicoaortic conduit, or AAC) to create a double-outlet left ventricle (LV) has been successfully employed to treat a variety of congenital LV outflow obstructions as well as aortic stenosis in patients where the traditional valve replacement surgery was impossible or difficult. In the general practice of cardiac surgery, the AAC insertion operation, with or without cardiopulmonary bypass, was displaced by direct aortic valve replacement that is more straightforward and associated with less post-surgical complications. For most surgeons today, AAC implantation is not a common operation and is of historical interest only.

While there have been several techniques described, the most commonly employed method of AAC implantation is the lateral thoracotomy (muscle-sparing and less invasive type of thoracic surgery) with the attachment of the AAC outflow duct to the descending aorta. Other surgical techniques include a median sternotomy approach with attachment of the distal limb of the AAC to the ascending aorta, to the aortic arch, or to the intra-abdominal supra-celiac aorta.

For example, the thoracic aorta and the left ventricle apex can be exposed through a left lateral thoracotomy, and a trocar is pierced through the apex and into the left ventricle. While the apical connector of the AAC is still spaced apart from the apex, the sutures that will fix the connector to the apex are threaded through a cuff on the connector and through the apex in a matching pattern. The cuff is set back from the end of the connector by 1-2 centimeters to allow the end of the connector to extend through the heart muscle and into the left ventricle. Once the sutures are in place, a ventricular coring device is used to remove a core of ventricular muscle, and the pre-threaded sutures are then pulled to draw the connector into the opening until the cuff comes to rest on the apex. The sutures are tied off, and additional sutures may be added. Either before or after this procedure, the opposite end of the connector is attached to a valved conduit which terminates at the aorta. The current techniques and technology available to perform AAC insertion were originally designed to be performed on-pump, either with the heart stopped. Since then, off-pump surgeries on a beating heart were successfully performed. It is appreciated that novel, less invasive surgical techniques and instruments will emerge in the future to establish a conduit between the apex of the heart and a major artery, such as an aorta.

### SUMMARY OF THE INVENTION

It is indisputable that reduction of diastolic volume and pressure in the LV of the heart will benefit CHF patients in the late stage of the disease. This benefit can be expected to manifest in the reduction of pulmonary edema in the short term and in the reversal of the progressive dilation and remodeling of the heart, improvement of the heart pumping ability and quality of life for patients in the long term. Many patients would benefit from the decreased LV diastolic volume. Existing therapies for that purpose, such as drugs, LVADs and heart constraints met with limited success.

It would be advantageous to reduce pressure and volume of the LV during heart diastole and therefore reduce the drive of the heart to dilate and pulmonary edema resulting in shortness of breath. Further, it would be advantageous to achieve diastolic unloading in an active controllable, reversible and gradual fashion that requires reasonable surgery. The methods and systems disclosed herein controllably pump small amount of blood volume out of the LV of the heart during heart diastole. The observation by the inventors that in the majority of patients with dilated hearts reduction of diastolic volume (preload) of the LV is followed by the proportional reduction of systolic volume while maintaining blood pressure and cardiac output. Until now there were no practical known ways to reduce diastolic volume in a reversible controllable way without major surgery.

Reducing Left Ventricular End Diastolic Pressure (LVEDP) reduces the back pressure into the pulmonary venous system of the lungs, minimizing shortness of breath. Shortness of breath is the result of fluid moving across the capillaries of the lung into the interstitial or alveolar spaces. This fluid impairs the transfer of oxygen, resulting in arterial desaturation and the feeling of shortness of breath. In the absence of dexoygenation, simply having fluid in the interstitial space of the lung activates receptors that generate the feeling of shortness of breath. Transudation of fluid into the lung occurs at the capillaries. That is why high left sided (pulmonary venous) pressures lead to shortness of breath and high right sided (pulmonary arterial) pressure do not. For this reason the discharge of blood in the disclosed preferred embodiment is into the right atrium of the heart. Patients with pure right sided heart failure from such things as increased pulmonary resistance can develop large amounts of fluid overload but result in acities (abdominal fluid) and leg edema. Increase pulmonary resistance occurs at the pulmonary arteriolar level that is pre-capillary. Thus, the capillaries are never exposed to the high pressures. Conversely, during diastole, the pulmonary veins, left atrium and left ventricle all see the same pressure. In other words, LVEDP is transmitted directly back to the capillaries in the lung.

In a given cardiac cycle (corresponding to one “beat” of the heart), the two atria contract, forcing the blood therein into the ventricles. A short time later, the two ventricles contract, forcing the blood therein to the lungs (from the right ventricle) or through the body (from the left ventricle). Meanwhile, blood from the body fills the right atrium and blood from the lungs fills the left atrium, waiting for the next cycle to begin. A healthy adult human heart may beat at a rate of 60-80 beats per minute (bpm) while at rest, and may increase its rate to 140-180 bpm when the adult is engaging in strenuous physical exercise, or undergoing other physiologic stress.

Every heartbeat cycle consists of two components: diastole and systole. Systole occurs when electrical impulse triggers the heart to contract. The left and right atria contract at nearly the same time pumping blood into the left and right ventricle. Systole continues as the right and left ventricle contract, pumping blood to the lungs and body, several tenths of a second after the right and left atria have contracted. Diastole occurs when the heart is relaxed and not contracting. During diastole, blood fills each of the atria and begins filling the ventricles. Systole and diastole continuously alternate as long as the heart continues to beat. The purpose of the invention is to unload the heart during diastole.

To address CHF, many types of cardiac assist devices have been developed. Traditional cardiac or circulatory assist device is one that aids the failing heart by increasing or supplementing its pumping function. Commonly used circulatory assist device employs a full or partial prosthetic connected between the heart and the aorta, one example of which is commonly referred to as a LVAD or Left Ventricular Assist Device. The LVAD comprises a pump and associated valves that draws blood directly from the apex of the left ventricle and directs the blood to the aortic arch, bypassing the aortic valve. In this application, the left ventricle stops functioning and does not contract or expand. The left ventricle becomes, in effect, an extension of the left atrium, with the LVAD taking over for the left ventricle. The HeartMate LVAD made by Thoratec Corporation (Pleasanton, Calif.) is the most common typical traditional LVAD. Although different designs of LVADs were proposed, they all assist the heart by supplementing or replacing systolic function of the heart. These pumps pump support and unload LV of the heart by pumping 4 to 10 liters/min of blood from LV into arterial system of the patient during heart systole or both heart systole and diastole. All existing LVADS discharge blood into patient's aorta or other parts of the arterial system with the intention of increasing cardiac output of the heart.

As noted above, devices that reduce the volume of the heart by reducing or preventing an increase the heart diameter can improve systolic heart function as the mechanical function of the heart is better at smaller heart diameters. Similarly, LVADs can reduce the work of the heart to a significant degree by draining blood from the heart's ventricle into the LVAD, reducing LVEDP and the stress on the heart.

Unfortunately, it is possible to reduce the LVEDP too much leading to such problems as low blood pressure, fainting, vital organ dysfunction and even death. Using a simple analogy, the contraction of the heart is similar to the recoil or “snap” of a rubber band. In order to be able to get a rubber band to recoil, it needs to be stretched from its minimal resting length. The further it is stretched, the more it recoils or “snaps”. However, if the rubber band is stretched excessively, it may either break or change it properties and lose its ability to recoil effectively.

Similar to a rubber band, the heart needs to be stretched, or filled to an adequate LVEDP during diastole, to allow it to “snap back”, or contract sufficiently to pump blood from of the heart during systole. The major function of an LVAD is its ability to fully drain all of the blood entering the heart and pump this blood into the arterial circulation to support the vital organs. While extremely beneficial in terms of providing diastolic unloading of the heart, LVADs remove so much blood from the heart that the amount of blood remaining in the heart during LVAD operation is insufficient to allow the heart to generate any blood flow on its own. Further, LVAD placement requires major surgery, is associated with a high risk of infections and strokes and is very costly to the health care system.

The inventors broke away from the tradition of complete diastolic unloading and total replacement of providing systolic blood flow and proposed a counterintuitive approach of partial heart unloading and reduction of diastolic heart size leading to an improvement in overall heart function. In an embodiment of the invention, a blood pump is used during heart diastole to remove a small amount of blood volume from the LV cavity. In one proposed embodiment, counterintuitively, the device returns this blood not to the high pressure arterial system to maintain or increase cardiac output but instead to the lower pressure venous system (possibly during a subsequent ventricular systole) to shift blood volume from the arterial to venous vasculature.

While the effects of under and overstretching described in the rubber band example above illustrate this issue, in reality, the amount of force generated by a muscle in general, and heart muscle specifically, is uniquely sensitive to the resting, or diastolic, stretch of the muscle immediately before it contracts. It is well know that the highest force of contraction of heart muscle occurs if the resting stretch of the heart results in the optimal overlap of the two proteins (called actin and myosin) responsible of contraction of heart muscle. Clinically, optimal blood pumping by the heart occurs when the heart is filled to the optimal LVEDP during diastole.

The clinical course of CHF is characterized by a slow, progressive deterioration in the ability of the heart to pump a sufficient amount of blood to fully maintain vital organ function. The body uses the just described ability to improve the pumping function of the heart by increasing LVEDP to its advantage. Though the body's intrinsic compensatory mechanisms (e.g., increasing neurohormal and sympathetic nervous system activity), the body directs the kidneys to retain extra fluid volume. In the earlier stages of CHF, this increase in fluid volume increases diastolic filling or LVEDP and make the heart again able to pump sufficient blood to support the vital organs. However, in the later stages of CHF, further increases in LVEDP do not result in a significant increase in the heart's pumping ability. This non-linear relationship between the diastolic filling of the heart (represented on the x-axis by LVEDP) and its pumping ability (represented on the y-axis by cardiac output) is illustrated by the well known Frank-Starling curve. Clearly, there is a flattening of curve, thus increase in cardiac output, once the LVEDP exceeds 20 mmHg. Moreover, the symptoms of shortness of breath and lung congestion commonly occur in Chronic CHF patients once the LVEDP exceeds 22-25 mmHg as these high pressures drive transudation of fluid from the pulmonary capillaries into the lung tissue, impairing oxygenation.

It is clear from the well known Frank-Starling curve that it is possible to reduce LVEDP without impairing the cardiac output and maintaining adequate vital organ perfusion. Further, reducing LVEDP will reduce the diastolic size of the heart and prevent shortness of breath and lung congestion from high pulmonary pressures.

Thus, an advantage of our proposed approach is to remove a sufficient amount of blood to allow a clinically relevant reduction in LVEDP leading to a reduction in diastolic heart size and/or prevention of remodeling yet assuring sufficient diastolic filling of the heart to allow the heart to pump enough blood during systole to support the needs of the body.

Adding Aortic Compliance:

It would be advantageous to create a less invasive solution for cardiac surgery to enable implantation of a medical device in a human that increases the compliance of the aorta. It is generally expected that the increased aortic compliance decreases the energetic cost of cardiac ejection at the same level of cardiac performance (cardiac output), thus reversing or slowing the deterioration of patients with heart failure. In addition, it is also desired to reduce the resistance of the aorta and the aortic valve. All of the above could potentially benefit large number of cardiac disease patients with heart failure, hypertension, diabetes, vascular disease and others.

The inventors propose to use the existing relatively less invasive valved apicoaortic conduit (also called AAC) techniques and technologies with a novel addition of a complimentary compliance chamber to supplement the compliance of the patient's own aorta. One preferred embodiment of the invention is a novel device that is similar to a common AAC device, such as for example the Carpentier-Edwards Bioprosthetic Valved Conduit made by Edwards Lifesciences Corporation (Irvine, Calif.), but in addition includes a compliance chamber between the one-way (unidirectional) valve and the aorta. The invented device is further called apicoaortic compliance conduit or AACC.

It is understood that while that particular embodiment shows the patient's aorta as a site of blood drainage, it can be any of a number of large arteries such as a femoral artery, a renal artery or a subclavian artery.

One described preferred embodiment has following advantage over known devices that increase aortic compliance:

A. It connects to the apex of the heart. This surgery is less invasive than other thoracic surgeries, can be performed on a beating heart, utilizes existing skills of surgeons (many popular Left Ventricular Assist Devices or LVADs are connected that way), and is reversible.

B. Providing additional path for blood to exit the LV and the second valve also results in the reduced outflow resistance in addition to the reduced compliance.

C. There may be situations in which all of the cardiac output would ideally flow through the AACC. Conversely, there may be situations in which the total blood flow should be split between the AACC and normal anatomic pathways for blood flow. The diameter, length and compliance of the AACC can be chosen so that a predetermined percentage of total cardiac output is ejected via the AACC and via the patient's own aortic valve.

### SUMMARY OF THE DRAWINGS

A preferred embodiment and best mode of the invention is illustrated in the attached drawings that are described as follows:

FIG. 1 illustrates the surgical implantation of the diastolic unloading pump and blood conduits.

FIGS. 2A, 2B, 2C and 2D illustrate alternative designs of the diastolic unloading pump.

FIG. 3 illustrates the design of the pump controller.

FIG. 4 illustrates diastolic unloading with blood flow and pressure during one heart beat.

FIG. 5 illustrates an algorithm of diastolic unloading control.

FIG. 6 illustrates added aortic compliance.

FIGS. 7 and 8 illustrates design and operation of one embodiment of the aortic compliance during the heart cycle.

### DETAILED DESCRIPTION OF THE INVENTION

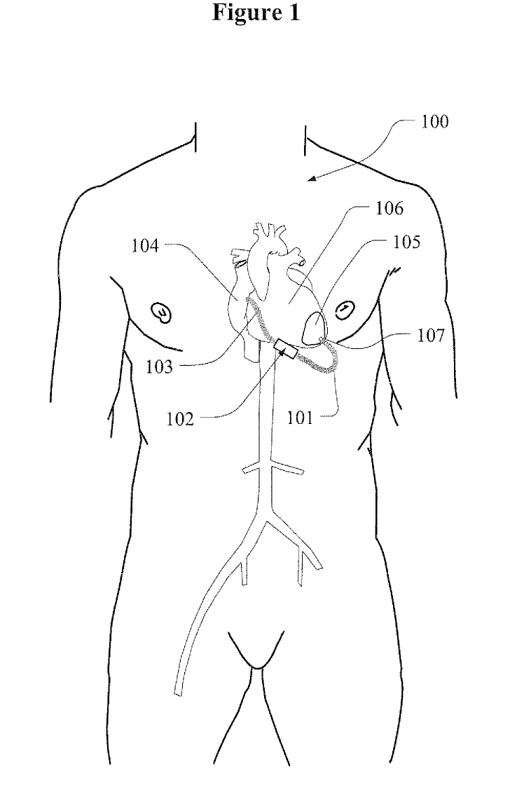

FIG. 1 illustrates key mechanical elements of one preferred embodiment of the invention. The patient 100 received an implanted diastolic unloading pump (blood pump) 102. The inlet blood duct 101 is attached to the apex 107 of the heart 106 and is in fluid communication with the left ventricle cavity (LV) 105. It is attached by suture or by other known cardiac surgery means to the heart muscle. The inlet duct allows passage of blood from the LV to the blood pump 102 that is connected to the outlet duct 103 for the return of blood. The outlet duct 103 is connected and is in fluid communication with the right atrium 104 of the heart 106. It is understood that many other connection sites for inlet and outlet ducts can be used by surgeons depending on the particular requirements of surgery. The entire device assembly including inlet and outlet ducts may be contained within the thoracic cavity to simplify surgery. Great arteries and veins such as aorta, vena cava, right atrium and right atrial appendage can also be selected to receive blood flow from the outlet duct. The pump 102 is controlled by the controller electronics (Not Shown) to pump blood from the LV 105 into the right atrium 104 (or other blood cavity such as the thoracic aorta) during the heart diastole (relaxation period).

Every heartbeat cycle includes two components: diastole and systole. Systole occurs when electrical impulse triggers the heart to contract. The left and right atria contract at nearly the same time pumping blood into the left and right ventricle. Systole continues as the right and left ventricle contract, pumping blood to the lungs and body, several tenths of a second after the right and left atria have contracted. Diastole occurs when the heart is relaxed and not contracting. During diastole, blood fills each of the atria and begins filling the ventricles. Systole and diastole continuously alternate as long as the heart continues to beat. The purpose of the invention is to unload the heart during diastole.

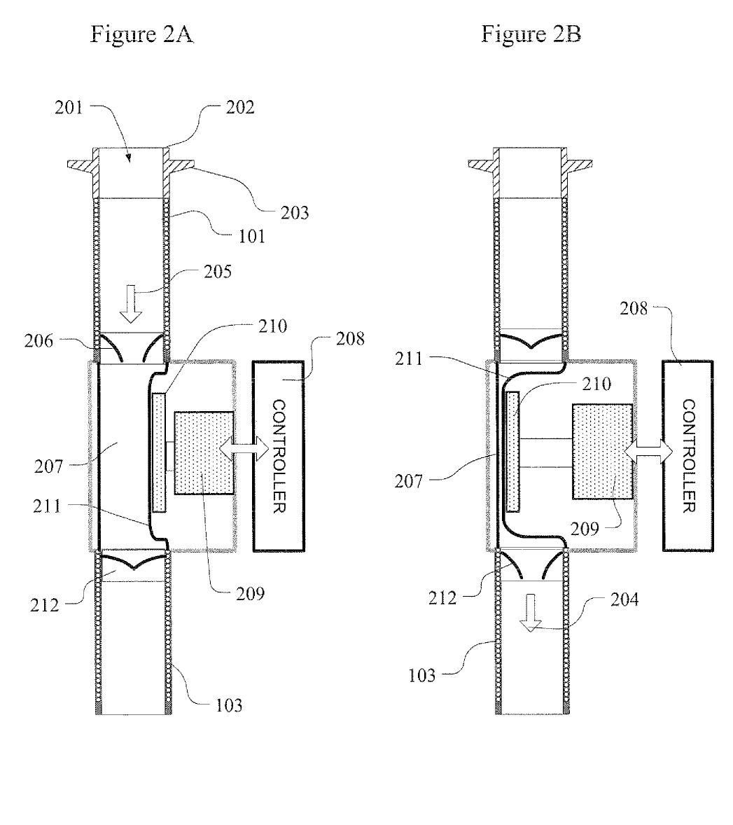

FIGS. 2A and 2B illustrate the schematic design and operation of one embodiment of the pump 102. This embodiment is a valved displacement pump. Pump operation is shown during diastole and systole of the heart. Blood inlet duct 101 is attached to the apical connector 201 to facilitate surgical attachment to the apex of the heart. Connector 201 is equipped with the cuff 203 for suturing to the heart apex muscle wall. The protruding proximal end 202 of the connector is designed to traverse the muscle wall and penetrate and enter the internal cavity of the LV of the heart. Outlet duct 103 can be sutured into the right atrial appendage of the heart or other appropriate location for blood return. Conduits 101 and 103 can be made of any material suitable for commercially available implantable conduits used to replace parts of a diseased aorta or other great vessels in adults and children. Conduits can be made of reinforced EPTFE, Dacron, Silicone or other durable, biocompatible polymer. Conduits can be 6 mm internal diameter to prevent clotting and assure low resistance to flow. Duct 101 can be reinforced with wire to prevent collapse if negative pressure is applied. Examples of blood conducting conduits and valves that can be adapted for the use with the invention are available from Edwards Lifesciences Corporation (Irvine, Calif.) such as the Carpentier-Edwards Bioprosthetic Valved Conduit or from Medtronic Inc., Minneapolis, Minn. such as is the Freestyle® aortic root bioprosthesis valve sleeve and the Hancock apical left ventricular connector. Apical LV connector incorporates a low porosity graft connected to a rigid (polypropylene) curved connector which is inserted into the left ventricular apex. Grafts are sized from 8 mm to 26 mm internal diameter. The Medtronic graft is intended to be anastamosed to the Hancock valved conduit.

FIG. 2A shows pump during heart diastole. Blood 205 flows through the apical connector 201 from the LV (Not Shown), through the opened inlet valve 206 into the into the pump compliance 207. Increase of the pump compliance volume determines the amount of blood removed from LV cavity during heart diastole. Controller 208 commands the pump actuator 209 that can be, for example, a solenoid or a liner motor to retract the compression plate 210 that is attached to the resilient diaphragm 211. As the diaphragm 211, which can be made of an elastic material such as silicone rubber, is retracted towards the actuator 209 and low pressure or suction is generated in the compliance 207. This suction opens the inlet valve 206, motivates blood to flow from the LV into the pump. Outlet valve 212 is closed since pressure in the outlet duct 103 (equal for example to right atrial pressure) is higher than the pressure in the compliance 207.

FIG. 2A shows pump during heart systole. Ejected blood 204 flows through the outlet duct 103 from compliance 207, through the opened outlet valve 212 into the right atrium of the heart or other appropriate blood return site. Decrease of the pump compliance volume determines the amount of blood removed from the compliance volume cavity during heart systole. Controller 208 commands the pump actuator 209 to advance the compression plate 210 that is attached to the diaphragm 211. As the diaphragm 211 is advanced away from the actuator 209 high pressures is generated in the compliance 207. This suction opens the outlet valve 212, motivates blood to flow from the pump into the right atrium. Inlet valve 206 is closed since pressure in the inlet duct 101 (equal for example to systolic LV pressure) is lower than the pressure in the compliance 207.

FIG. 2C illustrate the schematic design and operation of an alternative embodiment of the pump 102. This embodiment is a peristaltic pump. Peristaltic pump is substantially occlusive displacement pump and does not require valves. During the operation (diastole of the heart) pump rollers 222 are rotated by the motor actuator 221 controlled by the controller 208. The rollers squeeze blood 204 out of the elastic compressible tube segment 220 that connects inlet and outlet ducts. Design of peristaltic pumps using two, three or four rollers is well known in the art of blood pumping and does not require detailed description.

FIG. 2D illustrates the schematic design and operation of an alternative embodiment of the pump 102. This embodiment is an axial impeller pump. The impeller 226 is equipped with blades or fins that propel blood 204 when the impeller is rotated by the application of alternating electric current to electric magnets or coils 225. The controller 208 commutates the impeller as an electric motor and may accelerate it during diastole and slow it down during systole. The impelled can be supported by magnetic or blood lubricated bearings. Such designs are known in the field of blood pumps.

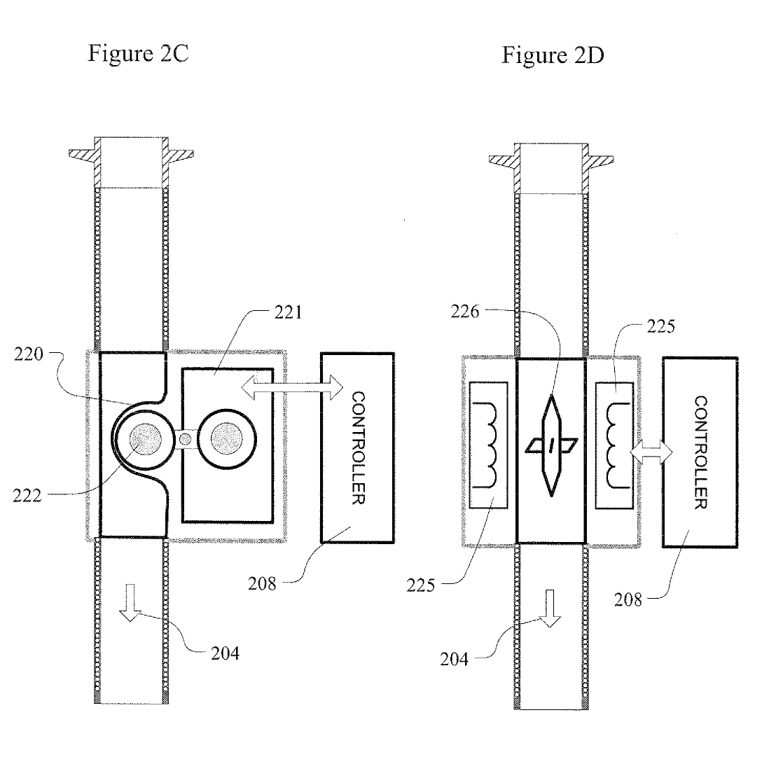

FIG. 3 illustrates the schematic and operation of the pump controller 208. Controller electronics is capable of receiving and processing physiologic information 301 from the patient such as ECG signal or blood pressure waveform in real time. Implantable sensors and amplifiers for ECG and pressure sensing are known in the art of pacemakers and other implantable electronic devices. Signals are converted into digital information by the by the Analog to Digital Converter ADC 302. CPU that can be an embedded software containing microprocessor 303 receives the information from ADC and implements control algorithms. Pump 102 is powered by power electronics 304 that is controlled by the software embedded in the CPU 303. Telemetry electronics 310 allows the operator to reprogram parameters of therapy such as pump flow and pump timing settings. Power to the controller electronics is supplied by the internal battery 305. The battery can be rechargeable. The implantable controller can also include the RF receiver electronics 306 that receives power from RF coil 309. The RF transmitter antenna (external coil) 308 emits RF that is received by implanted coil 309 to recharge battery 305. All these elements of the controller are known in the art of implantable medical devices such as ICDs, pacemakers, IPGs and VADS.

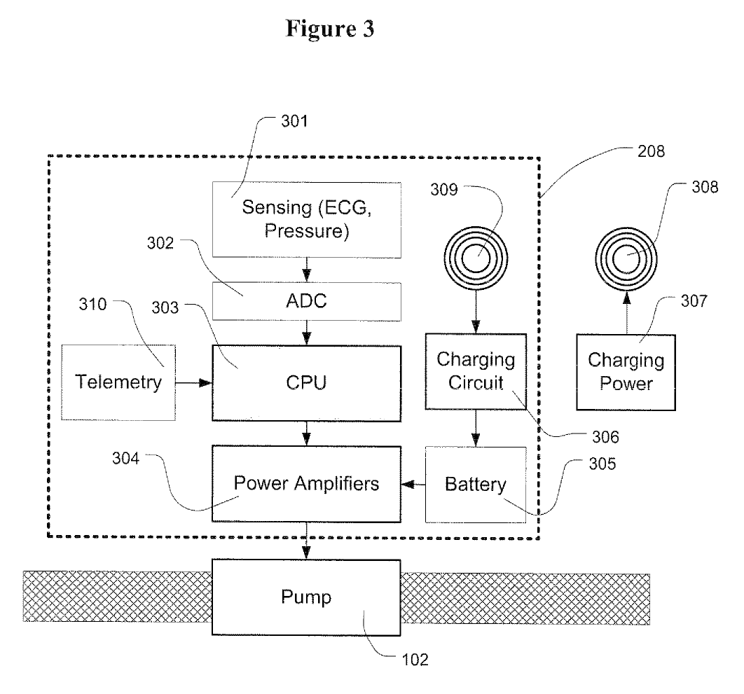

FIG. 4 shows the effects of diastolic ventricular unloading with the pump on vascular pressures of the patient during the heart cycle. Graph 400 shows that the pressure in the left ventricle (LV) chamber of the heart is at the highest level or systole 402 and the lowest level or diastole 403 for the entire heartbeat cycle. Aortic pressure 401 follows LV pressure during systole. Because of the low pressure during diastole, the LV fills with blood. The pressure in the aorta 401 (downstream of the left ventricle) is also at a relatively-low state, but not as low as the LV pressure. There is minimal electrical activity of the heart during the diastole rest state. LV End Diastolic Pressure LVEDP 405 is elevated (typically to 15-35 mmHg) in CHF patients compared to normal LVEDP of 5-15 mmHg. This elevated LVEDP is at the root of the progressive dilation of the heart and pulmonary edema (water in the lungs) caused by blood pressure reflected back from the LV to pulmonary capillaries that are permeable to water.

While the heart is in the diastole rest state, the heart muscle receives blood from the coronary arteries. This blood flow to the heart muscle is critical to sustaining the health of the heart. While pumping blood, the heart inhibits its own blood supply due to the contraction of the heart muscle. As the heart muscle contracts, coronary blood flow to the left ventricle chamber of the heart is throttled by the tense state of the heart muscle. Only after the heart relaxes, can blood flow into the heart muscle. The present invention improves blood flow to the heart muscle by assisting the relaxation of the heart muscle and reducing the downstream pressure for coronary perfusion. Under normal conditions, the distribution of coronary blood flow across the heart wall is uniform. The diastolic gradient from aorta to the heart during diastole favors coronary flow. However, in the failing heart, and especially with coronary artery decease, a substantially-reduced quantity of blood is delivered to the internal layers of the heart muscle. Flow to these layers of muscle occurs predominantly during diastole and depends on the driving coronary perfusion pressure gradient. Ventricular diastolic pressure is the downstream pressure for this gradient and inhibits flow in direct proportion to its level.

Trace 410 illustrates the electric activity of the heart. In one embodiment of the invention, the pump operation can be triggered by the R-wave 411 of the ECG signal 410 on a real-time basis. Ventricular contraction (systole) begins at point 411 which corresponds to the peak of the QRS complex, and continues until the T-wave 413. At point 411 the mitral (and bicuspid) valves close due to increase in ventricular pressure (as the ventricles contract). The closing mitral and biscupid valves produce the first heart sound that also can be potentially electronically detected and used to control ventricular unloading pump. The ventricular contraction forces blood into the aorta and an increase in both aortic and ventricular pressure is noted between points 411 and 413. As blood is pumped from the ventricles and carried away in the aorta, ventricular pressure drops. When the pressure drops below aortic pressure, the semilunar valves slam shut at point 413. Ventricular muscle repolarization begins at the end of the T-wave 413 and causes further decrease in ventricular pressure. Shortly after point 413 the ventricular pressure falls below atrial pressure and the mitral and bicuspid valves open. Atrial contraction 412 begins the middle of the P-wave and continues throughout the PR interval. The atrial pressure increasing as the atria contract. As blood is pumped into the ventricles the ventricular pressure also rises. The PR interval corresponds to the delay necessary for the ventricles to fill after atrial contraction. Note that the atrial repolarization wave (electrical impulse) is usually hidden by the QRS complex and atrial muscle relaxation occurs after the QRS complex and is accompanied by a decrease in atrial pressure.

Trace 420 illustrates synchronization of the pump flow to the heart cycle. Diastolic unloading pump flow 421 can be zero or relatively low 421 during systole (between points 411 and 413) and substantially increased 422 during diastole. Pump can be activated at a fixed delay of 400 to 600 ms following the detection of QRS 411 or immediately following detection of the T-wave 413. It is anticipated that that it may take the pump 50 to 200 ms to respond to the command signal. If the heart speeds up, then the triggering of the diastolic unloading also occurs more frequently to keep up with the heart. The heartbeat is sensed by ECG electrodes placed on the patient's heart or in proximity to the heart. The electrodes detect an electrical signal that is processed by an implanted electronic electrocardiographic instrument to generate an electrocardiogram (ECG) signal. The ECG signal has certain signature characteristics, such as the QRS wave 411 that indicates the onset of the systole phase and ventricular contraction. When the R wave in the ECG signal is detected, a time counter can be started to start pumping out the LV after a delay. The delay can be a variable stored in the CPU memory. A computer controller processes the ECG signal and detects the QRS wave using relatively-simple band-pass filtering techniques. The controller triggers the pump flow control, by activating a controller electronics associated with the pump. The controller can automatically adjust the duration of pumping and the delay.

Trace 430 illustrates the volume of LV cavity during an assisted and unassisted heart cycles. Volume is lowest 432 at the end of systole and largest 431 at the end of diastole. Comparing panels 400 (pressure) and 430 (volume) illustrates the effects of the invention of the LV performance. Line 404 (dashed) demonstrates reduction of diastolic LV pressure by pumping compared to unassisted pressure 403. Of particular importance is the reduction of LVEP 405 to LVEDP 406 that can be from 35 to 10 mmHg. Corresponding trace 430 illustrates changes of LV diastolic volume 431 (unassisted) that in a dilated heart can be 250 milliliters to lower 433 (assisted) volume that can be, for example, 245 milliliters. Difference between volume 431 and volume 433 approximately corresponds the volume of blood removed by the pump, that can be between 5 and 10 milliliters during the diastole of one single heart cycle. Volume can be pumped out during last 150 to 400 ms of the heart diastole. It is expected that removal of this relatively small volume of blood from the heart repeatedly on the beat-to-beat basis will result in the blood volume shift over time, unload the heart, reverse or arrest dilation and resolve pulmonary edema. Removal of blood can be implemented every heart beat or every second beat or at some other suitable rate that is acceptable for the patient.

While the heart is in the diastole rest state, the heart muscle receives blood from the coronary arteries. This blood flow to the heart muscle is critical to sustaining the health of the heart. While pumping blood, the heart inhibits its own blood supply due to the contraction of the heart muscle. As the heart muscle contracts, coronary blood flow to the left ventricle chamber of the heart is throttled by the tense state of the heart muscle. Only after the heart relaxes, can blood flow into the heart muscle. The present invention improves blood flow to the heart muscle by assisting the relaxation of the heart muscle. As the heart contracts to pump blood, the muscle fibers in the heart become tense to bind the layers of the muscle together. Releasing the tension in the heart muscle during the diastole period aids in expanding, i.e., relaxing the heart and the left ventricle. Under normal conditions, the distribution of coronary blood flow across the heart wall is uniform. The diastolic gradient from aorta to the heart during diastole favors coronary flow. However, in the failing heart, and especially with coronary artery decease, a substantially-reduced quantity of blood is delivered to the internal layers of the heart muscle. Flow to these layers of muscle occurs predominantly during diastole and depends on the driving coronary perfusion pressure gradient. Ventricular diastolic pressure is the downstream pressure for this gradient and inhibits flow in direct proportion to its level.

In the healthy heart, left ventricular diastolic pressure is in the range of 5-15 mm Hg and presents negligible opposition to coronary flow that is driven by a diastolic aortic pressure of 60-90 mm Hg. With coronary obstruction, this driving pressure gradient can be severely reduced as blood travels forward along a clogged artery. In addition, in the failing heart, the left ventricular diastolic pressure 200 is often elevated to 15 to 35 mm Hg over the pressure of a healthy heart. Under these circumstances, small changes in ventricular diastolic pressure become one of the primary determinants of flow in sub-endocardial (internal) layers of the heart muscle.

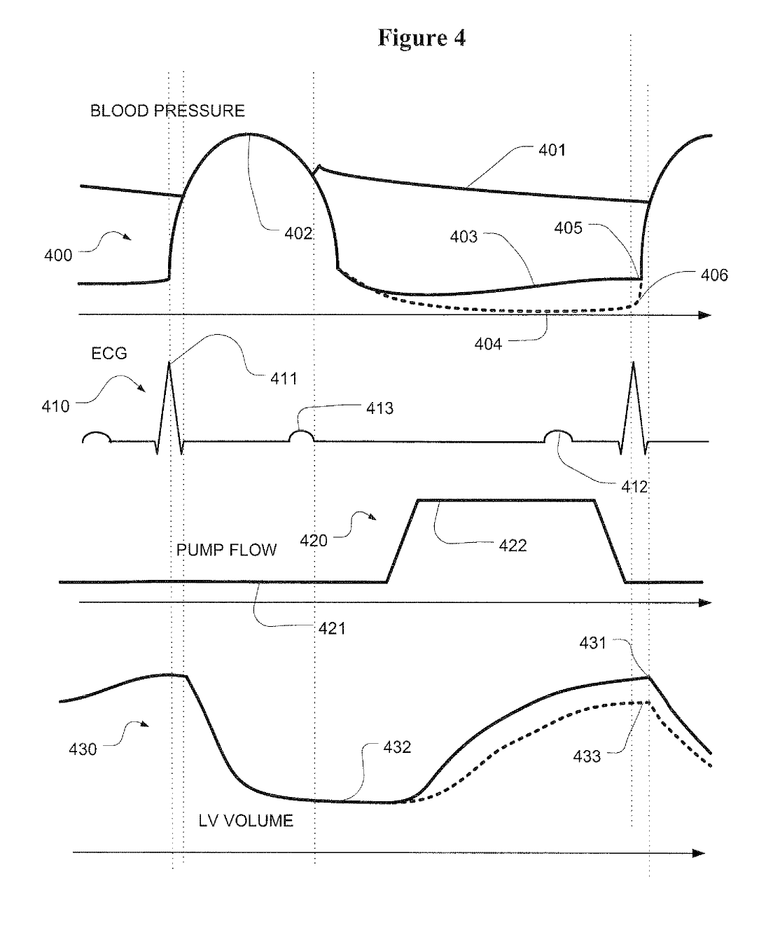

FIG. 5 illustrates an algorithm that can be embedded in the CPU of the embodiment of the invention. Heart cycle parameters are constantly monitored 501 from beat to beat to determine the duration of systole and diastole using implanted ECG or pressure sensors. After the QRS is detected (at beginning of the systole of the heart beat) counter is set to delay pumping 502 until the end of systole of the heart based on the known monitored heart rate. At the estimated beginning of diastole pump starts pumping blood from the LV 503. It is expected that the preprogrammed amount of blood will be pumped out or that pumping will continue until the desired LV pressure is reached 504. After the end of the preset diastolic pump assistance software waits for the next QRS detection 505.

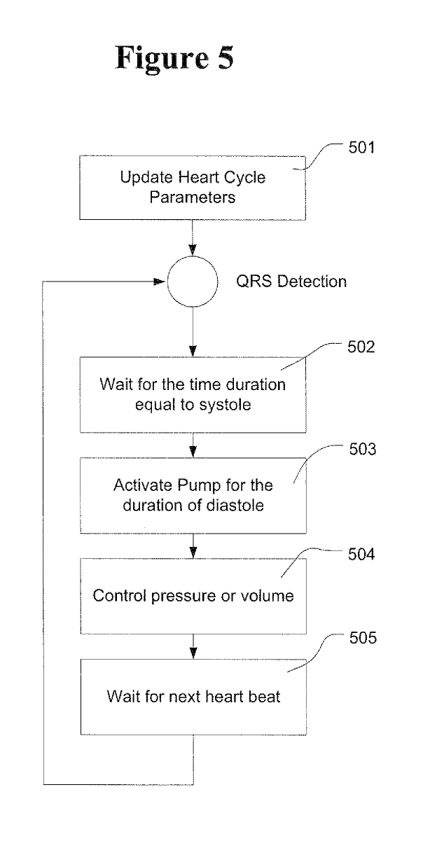

FIG. 6 illustrates key elements of one preferred embodiment of the apicoaortic compliance conduit or AACC invention.

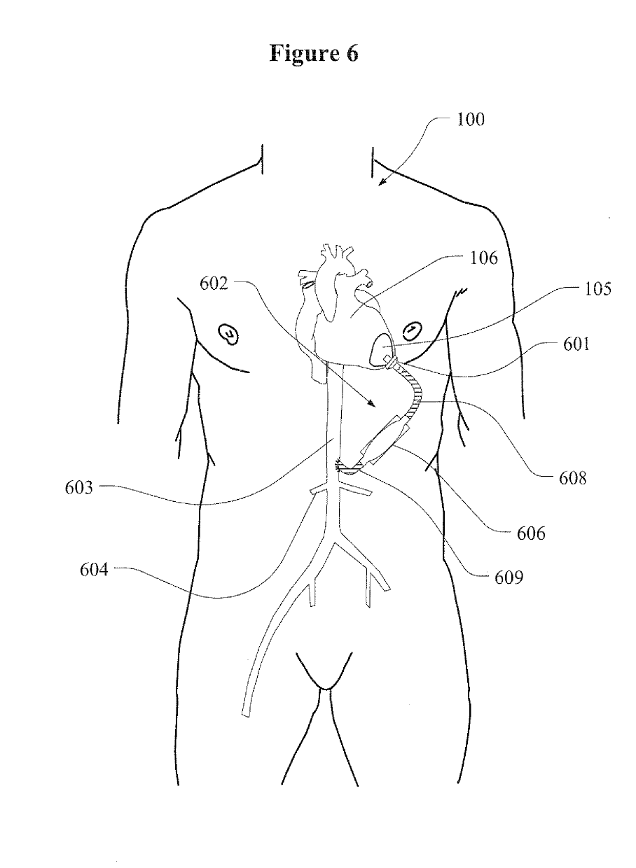

The patient 100 received an implanted AACC device 602. The apical connector 601 is attached to the apex of the heart 106 and penetrates into the left ventricle (LV) cavity 105. It is attached by suture or by other known cardiac surgery means. The connector is equipped with a valve (See FIG. 7). The valve allows passage of blood from the LV to the proximal (closer to the heart) AACC duct 608 when the heart contracts and prevents retrograde flow. The proximal duct 608 is attached to the compliance chamber 606. The compliance chamber 606 is attached to the distal duct 609 that is attached to the aorta 603. In the illustrated embodiment, the distal attachment is made just above the renal arteries 604. In this case, the device traverses the patient's diaphragm. It is understood that many other connection sites can be used by surgeons depending on the particular requirements of surgery. The entire AACC device including proximal and distal ducts may be contained within the thoracic cavity to simplify surgery. Great arteries other than aorta 603 such as aortic arch can also be selected to receive blood flow from the distal duct.

According to one embodiment of the invention, the invented AACC device consists as a minimum of the following elements in fluid communication:

1. Inflow connector attached to and receiving blood flow from the apex of the heart

2. One-way valve allowing blood flow away from the heart

3. Compliance element such as a compliance chamber

4. Outflow duct for connection to a great artery such as an aorta

It is understood that the outflow and inflow (proximal and distal) ducts as well as other elements of the invention can be mechanically combined into one assembly by a skilled engineer and look differently from the schematic drawings on FIGS. 6, 7 and 8.

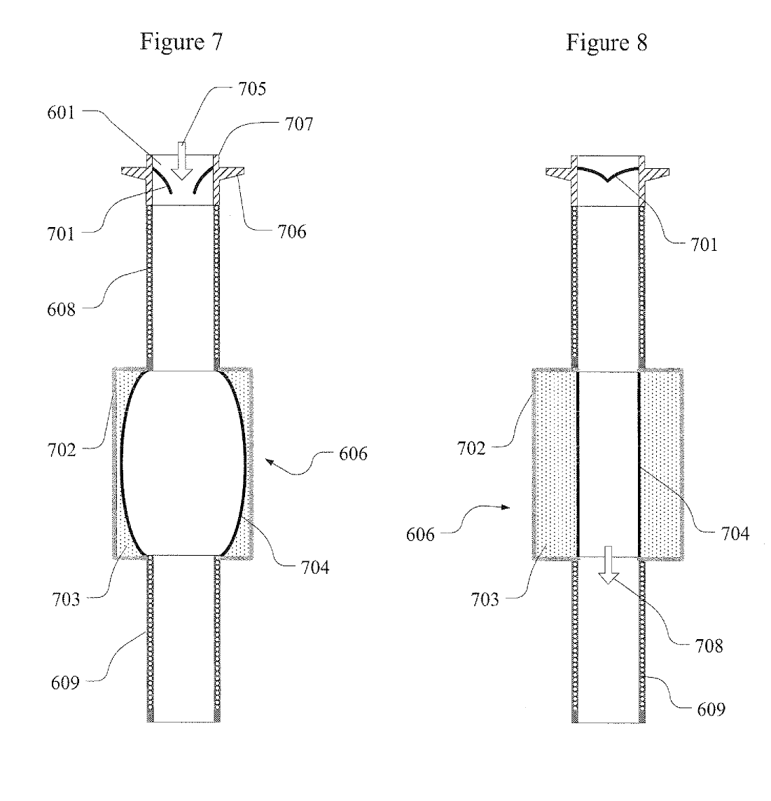

FIGS. 7 and 8 illustrate one embodiment of the AACC device 602. FIG. 7 shows the AACC immediately following the systole (ejection) part of the cardiac cycle or heartbeat.

Every heartbeat cycle consists of two components: diastole and systole. Systole occurs when electrical impulse triggers the heart to contract. The left and right atria contract at nearly the same time pumping blood into the left and right ventricle. Systole continues as the right and left ventricle contract, pumping blood to the lungs and body, several tenths of a second after the right and left atria have contracted. Diastole occurs when the heart is relaxed and not contracting. During diastole, blood fills each of the atria and begins filling the ventricles. Systole and diastole continuously alternate as long as the heart continues to beat.

On FIG. 7 the valve 701 inside the apical connector 601 is in the open position. Connector is equipped with the cuff 706 for suturing to the heart apex muscle wall. The protruding proximal end 707 of the connector is designed to traverse the muscle wall and penetrate and enter the internal cavity of the LV of the heart. The heart is ejecting blood 705 into the AACC forcing valve 701 to open. Proximal conduit 608 conducts blood flow to the compliance chamber 606. Conduits 608 and 609 can be made of any material suitable for commercially available implantable conduits used to replace parts of a diseased aorta or other great vessels in adults and children. Conduits can be made of reinforced EPTFE, Dacron, Silicone or other durable, biocompatible polymer. Blood ejected by the heart enters the compliance chamber 606. Outer walls of the compliance chamber 702 are made of a substantially inelastic material and form a rigid outer shell. Inside the rigid shell 702 is the chamber with elastic walls 704. Between the elastic walls 704 and inelastic walls 702 is compressible gas 703. During systole the elastic walls 704 stretch to accommodate the energy of the ejected blood 705. Gas 703 is compressed absorbing and storing the energy of the heart beat.

FIG. 8 shows the AACC during the diastole (rest) part of the cardiac cycle. Valve 701 is in the closed position. The heart is filling with blood preparing for the next systole (ejection). Energy stored during systole in the compressed gas 703 is being released. Elastic walls 704 of the compliance chamber 606 resume their relaxed position. Blood flow 708 is being propelled by the stored energy towards the distal conduit 609 and enters the body circulation.

Following elements of existing and known devices can be used to construct valve 701 and ducts 608 and 609 components of the AACC device with reasonably straightforward modifications:

Available from Edwards Lifesciences Corporation (Irvine, Calif.) is the Carpentier-Edwards Bioprosthetic Valved Conduit. The conduit is made from a porcine aortic valve that has been preserved and mounted on a flexible frame to prevent cracking. The frame is composed of a corrosion-resistant cobalt chromium metal alloy and silicone rubber covered with polytetrafluroethylene (PTFE) cloth. Commercially available valve diameter is 12 mm.

Available from Medtronic Inc., Minneapolis, Minn. is the Freestyle® aortic root bioprosthesis valve sleeve and the Hancock apical left ventricular connector. Low porosity graft connected to a rigid (polypropylene) curved connector which is inserted into the left ventricular apex. Sized from 8 mm to 26 mm internal diameter. This graft is intended to be anastamosed to the Hancock valved conduit.

Hancock valved conduits are low porosity grafts incorporating a Hancock standard porcine valve within the conduit. Sizes range from 12 mm up to 30 mm for right heart applications, 12 mm up to 26 mm for left heart applications. Hancock bioprosthetic valved conduits consist of an unstented porcine aortic valve, sutured into the center of a woven fabric conduit. The Hancock conduits are typically used for reconstruction of congenital or acquired cardiac and great vessel malformations or pathology.

The elastic walls 704 of the compliance chamber 606 can be made of a suitable biocompatible polymer resistive to fatigue such as a silastic silicone rubber. The inelastic walls 702 can be made of a metal such as titanium alloy or a polymer such as PEEK.

The illustrated compliance chamber is a cylinder traversed by the blood conduit along the central axis. It is intended to illustrate the principle, rather than to propose an engineering design. It is understood that there is any number of shapes that can be advantageous to design and fit to particular requirements of human anatomy. A skilled engineer can adapt the compliance chamber to a shape desired.

In the embodiment illustrated by FIGS. 7 and 8 the compliance chamber has a rigid external shell and a compressible gas compartment. It is understood that there are many other ways to construct a compliance chamber. A skilled engineer can use bellows, spring loaded pistons (such as in a car shock absorber) or a simple tube, pillow or sack made of an elastic compliant material. In the latter case (of a single wall chamber) the device design is simpler and more flexible but also more vulnerable to leaks, rupture, kinking, pinching by patient's muscle motion and compression by scar tissue after surgery. The double wall (with the rigid outer shell) design of the chamber can be expected to be safer and more reliable but also more complex and more difficult to implant. It is anticipated that a skilled engineer can select a material that is so strong and so resistive to pulsatile fatigue that the double wall design illustrated by FIGS. 7 and 8 will not be necessary.

Regardless of the engineering implementation of the invention it can be characterized as:

A medical device fully implantable in a body of a patient comprising the following elements:

1. a tubular connector inserted into an apex of the heart in fluid communication with left ventricular chamber of the heart,

2. a one-way valve configured to allow blood flow from the left ventricular chamber of the heart and prevent retrograde flow, and

3. a compliance element adapted for energy storage and release in fluid communication with the connector and the ventricular chamber of the heart, and the arterial system of the patient.

Table below represents possible design parameters for the compliance chamber. Material of the elastic wall of the compliance chamber is selected so, that in response to the normal heart pulse pressure change of approximately 40 mmHg (about from 90 mmHg diastolic to 130 mmHg systolic) the diameter of the chamber expands from 15 to 23 mm. If the cylindrical elastic chamber length is 140 mm, this increase results in approximately 33 ml of blood stored in the compliance. During diastole this volume is ejected by the recoil of the elastic wall into the arterial system of the patient.

| | | |
|---|---|---|
| Diastolic Diameter (contracted) | mm | 15 |
| Length of the Compliance Chamber | mm | 140 |
| Diastolic Volume | ml | 25 |
| Systolic Diameter (expanded) | mm | 23 |
| Systolic Volume | ml | 58 |
| Stroke Volume (Volume Change) | ml | 33 |

It can be generally expected that the additional volume of blood stored by the arterial compliance chamber as a result of elastic expansion in response to the pulse pressure change will be in the range of 20-50 mL.

The invention has been described in connection with the best mode now known to the applicant inventors. The invention is not to be limited to the disclosed embodiment. Rather, the invention covers all of various modifications and equivalent arrangements included within the spirit and scope of the appended claims.

What is claimed is:

1. A method to assist a failing heart of a patient comprising:
detecting a diastole of the heart;
pumping blood from a left ventricle of the heart during the diastole;
reducing the pumping of blood during a systole of the heart, and
repeating the steps of detection, pumping and reducing pumping during several heart beats

2. A method as in claim 1 where the pumped blood is returned to a venous system of the patient.

3. A method as in claim 1 where the step of pumping is executed during a latter half of a diastole period of the heart.

4. A method as in claim 3 wherein the step of pumping is executed during a period of 150 to 500 milliseconds at an end of the diastole period.

5. A method as in claim 1 where pumping includes removing 4 to 10 milliliters of blood during the diastole.

6. A method as in claim 1 where the detecting the diastole includes detecting a QRS of the heart.

7. A method as in claim 6 further comprising delaying the pumping following the detection of the QRS.

8. A method for assisting a failing heart of a patient comprising:
implanting a tubular connector to an apex of the heart in fluid communication with a left ventricular chamber of the heart,
implanting a one-way valve configured to allow a blood flow from the left ventricular chamber of the heart and prevent retrograde flow, and
implanting a compliance element adapted for energy storage and release in fluid communication with the connector and the ventricular chamber of the heart, and the arterial system of the patient.

9. A method as in claim 8 where the blood flow is returned to the aorta of the patient substantially during a diastole of the heart.

10. A system to assist a failing heart, the system comprising:
an implantable tubular connector adapted to provide fluid communication between an apex of the heart and a left ventricular chamber of the heart;
a one-way valve connected to the connector, and
a controller operating the one-way valve to allow a blood flow from the left ventricular chamber and prevent retrograde flow.
